(19) United States
(12) Patent Application Publication
(10) Pub. No.: US 2025/0152489 A1
(43) Pub. Date: May 15, 2025

(54) COSMETIC COMPOSITION

(71) Applicant: SHISEIDO COMPANY , LTD., Tokyo (JP)

(72) Inventors: Lethu NGUYEN, East Windsor, NJ (US); Alicja MIKULKO, East Windsor, NJ (US)

(73) Assignee: SHISEIDO COMPANY , LTD., Tokyo (JP)

(21) Appl. No.: 18/715,416

(22) PCT Filed: Dec. 1, 2022
(86) PCT No.: PCT/IB2022/061669

§ 371 Date: May 31, 2024

Related U.S. Application Data
(60) Provisional application No. 63/285,175, filed on Dec. 2, 2021.

(51) Int. Cl. A61K 8/37; A61K 8/06; A61K 8/26; A61K 8/27; A61K 8/29; A61K 8/31; A61Q 17/04

(52) U.S. Cl. CPC A61K 8/375; A61K 8/064; A61K 8/26; A61K 8/27; A61K 8/29; A61K 8/31; A61Q 17/04

(57) ABSTRACT

Disclosed is a water-in-oil type emulsified cosmetic composition that includes (a) at least two polyglycerol-type surfactants; (b) a solvent comprising at least one polar oil and at least one non-polar oil; and (c) water, which constitutes no more that 40% by mass of the composition.

### CROSS-REFERENCE TO RELATED APPLICATIONS

The present application claims priority under 35 U.S.C. § 371 to International Patent Application No. PCT/IB2022/061669, filed on Dec. 1, 2022, which is a Non-Provisional of U.S. Provisional Patent Application No. 63/285,175 filed Dec. 2, 2021. The contents of these applications are hereby incorporated by reference in their entireties.

### FIELD

The present disclosure relates in general to cosmetic compositions, such as water-in-oil cosmetic compositions.

### SUMMARY

One embodiment is a water-in-oil type emulsified cosmetic composition comprising: (a) at least two polyglycerol-type surfactants; (b) a solvent comprising at least one polar oil and at least one non-polar oil; and (c) water, which constitutes no more that 40% by mass of the composition.

### FIGURES

FIG. 1 presents plots of storage modulus (G′) and loss modulus (G″) versus oscillation stress for the composition of Example 1 (triangles) and the control composition of Comparative Example 1 (squares).

FIG. 2 presents plots of viscosity versus shear rate for the composition of Example 1 (triangles) and the control composition of Comparative Example 1 (squares). Table 5 shows shear viscosity values at 4 l/s and 90 l/s.

### DETAILED DESCRIPTION

Unless otherwise specified “a” or “an” means one or more.

As used herein, the term “about” placed before a specific numeric value may mean±20% of the numeric value; ±18% of the numeric value, ±15% of the numeric value; ±12% of the numeric value; ±8% of the numeric value; ±5% of the numeric value; ±3% of the numeric value; ±2% of the numeric value; ±1% of the numeric value or ±0.5% of the numeric value.

All content information for ingredients of compositions expressed as percent (%) refers to percent (%) by mass, relative to the total mass of the composition, unless specified otherwise.

As used herein, the expression “water-in-oil composition” and related expressions refers to an water-in-oil type emulsified composition in which an aqueous (water) phase is dispersed in an oil phase.

As used herein, the expression “microplastic particles” refers to particles containing a solid polymer, to which additives or other substances may have been added, and where ≥1% w/w of particles have (i) all dimensions 0.1 μm≤x≤5 mm, or (ii), for fibres, a length of 0.3 μm≤x≤15 mm and length to diameter ratio of >3.

The expression “particles containing a solid polymer” refers either (i) a particle of any composition with a continuous solid polymer surface coating of any thickness or (ii) particles of any composition with a solid polymer content of ≥1% w/w.

1,4-dioxane and ethylene oxide impurities of polyethylene glycol (PEG)(surfactants may present concerns for satisfying “clean beauty” requirements, such as safety, non-toxicity and transparency in labeling, for cosmetic products.

Some consumers have also environmental concern about a low biodegradability of silicone. Many water-in-oil type emulsified cosmetic products use bentonite-gel systems with PEG surfactants and/or silicone solvents. Eliminating PEG surfactants and silicone solvents can often negatively affect stability of cosmetic product and/or its sensorial properties.

In addition, many water-in-oil cosmetic compositions may have their oil phase separated over time and need therefore to be shaken up before the use. Furthermore, a color of stored water-in-oil cosmetic products may change due to pigment migration and agglomeration within a composition of the product.

The present disclosure provides a water-in-oil type emulsified composition, which may have improved stability. Specifically, the present composition may avoid oil phase separation over a period of at least one week, at least two weeks, or at least weeks at room temperature, or at an elevated temperature such as 45° C. or 50° C.

The present composition may also avoid color change due to pigment migration. The present composition may maintain sensory values, such as creaminess and ease of application. In many embodiments, the present composition may contain no PEG surfactant.

In some embodiments, the present composition may contain a silicone, such as a silicone solvent. Yet in some embodiments, the composition may be free of any silicones, such as silicon solvents.

In some embodiments, the present composition may contain a hydrocarbon, such as an alkane solvent.

In some embodiments, the present composition may contain an ester, such as a medium chain triglyceride (MCT) solvent.

In some embodiments, the present composition may be free of synthetic polymers.

In some embodiments, the present composition may be free of any polyethylene glycol containing ingredients, such as PEG surfactants, any polymeric ingredients, such as (meth)acrylates or (meth)acrylamide; and/or any silicones, such as silicone solvents.

In some embodiments, the composition may be free of any microplastic particles as defined above. Therefore, the present composition may qualify within “clean beauty” cosmetics category.

The present water-in-oil type emulsified composition includes (a) at least two polyglycerol-type surfactants; (b) a solvent comprising at least one polar oil and at least one non-polar oil; and (c) water, which constitutes no more that 40% by mass of the composition.

In some embodiments, the present water-in-oil type emulsified composition may include (a) at least two polyglycerol-type surfactants; (b) a solvent comprising at least one polar oil and at least one non-polar oil; (c) water, which constitutes no more that 40% by mass of the composition and (d) a thickener.

In some embodiments, the present water-in-oil type emulsified composition may include (a) at least two polyglycerol-type surfactants; (b) a solvent comprising at least one polar oil and at least one non-polar oil; (c) water, which constitutes no more that 40% by mass of the composition, (d) a thickener and (e) at least one pigment or UV filter.

The present composition may be used in a number of color cosmetics products. In addition, applications of the present composition may include skin care products, sun care products, deodorants or hair products. In some embodiments, the composition may be a lip composition, such as a lipstick or lip balm composition. In some embodiments, the composition may be a blush composition. In some embodiments, the composition may be a foundation composition, such as a solid foundation composition. In some embodiments, the composition may be a concealer composition. In some embodiments, the composition may be a complexion composition. In some embodiments, the composition may be an eyebrow composition, such as a solid or liquid eyebrow composition. In some embodiments, the composition may be an eyeshadow composition. In some embodiments, the composition may be a mascara composition.

In some embodiments, the cosmetic composition may be used alone. In other words, the composition may be applied alone, without another composition, to a keratinous surface or substrate, such as skin, e.g. lips, eyelids, face, or hair, of a subject, such as a human.

In some embodiments, the composition, which may be a cosmetic composition, may be used together with another product, for example, a top coat, a primer, or a powder.

As used herein, the term “water-in-oil type emulsified composition” refers to a composition in which an aqueous phase is dispersed in an oil phase.

### Water

Water, which is a component of the aqueous phase of the composition, may constitute no more or less than 40% by mass or no more of less than 39% by mass or no more or less than 38% by mass or no more or less than 37% by mass or no more or less than 36% by mass of the composition. In some embodiments, a content of water in the composition may be from 15 mass % to 40 mass % or from 20 mass % to 40 mass % or from 25 mass % to 39 mass % or from 30 mass % to 38 mass % or any value or subrange within these ranges.

### Polyglyceryl Surfactants

The composition contains at least two polyglyceryl surfactants, which may be for example, dissolved in the oil phase.

At least two polyglyceryl surfactants may be selected from polyglyceryl 2 surfactants, polyglyceryl 3 surfactants, polyglyceryl 4 surfactants, polyglyceryl 5 surfactants, polyglyceryl-6 surfactants, polyglyceryl 7 surfactants, polyglyceryl 8 surfactants, polyglyceryl 9 surfactants, polyglyceryl 10 surfactants, polyglyceryl 11 surfactants, and polyglyceryl 12 surfactants.

Exemplary polyglyceryl surfactants include, but not limited to, Polyglyceryl-4 Caprate, Polyglyceryl-2 Caprate, Polyglyceryl-4 Caprylate, Polyglyceryl-6 Caprylate, Polyglyceryl-6 Caprate, Polyglyceryl-4 Caprylate/Caprate, Polyglyceryl-6 Caprylate/Caprate, Polyglyceryl-3 Cocoate, Polyglyceryl-4 Cocoate, Polyglyceryl-10 Decalinoleate, Polyglyceryl-10 Decaoleate, Polyglyceryl-10 Decacasterate, Polyglyceryl-3 Dicaprate, Polyglyceryl-3 Dicocoate, Polyglyceryl-10 Didecanoate, Polyglyceryl-2 Diisostearate, Polyglyceryl-3 Diisostearate, Polyglyceryl-10 Diisostearate, Polyglyceryl-4 Dilaurate, Polyglycerin-2 Dioleate, Polyglyceryl-3 Dioleate, Polyglyceryl-6 Dioleate, Polyglyceryl-10 Dioleate, Polyglyceryl-6 Dipalmitate, Polyglyceryl-10 Dipalmitate, Polyglyceryl-2 Dipolyhydroxystearate, Polyglyceryl-2 Distearate, Polyglyceryl-3 Distearate, Polyglyceryl-6 Distearate, Polyglyceryl-10 Distearate, Polyglyceryl-10 Heptaoleate, Polyglyceryl-10 Heptastearate, Polyglyceryl-6 Hexaoleate, Polyglyceryl-10 Hexaoleate, Polyglyceryl-2 Isopalmitate, Polyglyceryl-2 Isostearate, Polyglyceryl-4 Isostearate, Polyglyceryl-5 Isostearate, Polyglyceryl-6 Isostearate, Polyglyceryl-10 Isostearate, Polyglyceryl-2 Laurate, Polyglyceryl-3 Laurate, Polyglyceryl-4 Laurate, Polyglyceryl-4 Laurate/Sebacate, Polyglyceryl-4 Laurate/Succinate, Polyglyceryl-5 Laurate, Polyglyceryl-6 Laurate, Polyglyceryl-10 Laurate, Polyglyceryl-3 Myristate, Polyglyceryl-10 Myristate, Polyglyceryl-2 Oleate, Polyglyceryl-3 Oleate, Polyglyceryl-4 Oleate, Polyglyceryl-5 Oleate, Polyglyceryl-6 Oleate, Polyglyceryl-8 Oleate, Polyglyceryl-10 Oleate, Polyglyceryl-3 Palmitate, Polyglyceryl-6 Palmitate, Polyglyceryl-10 Pentalaurate, Polyglyceryl-10 Pentalinoleate, Polyglyceryl-4 Pentaoleate, Polyglyceryl-10 Pentaoleate, Polyglyceryl-3 Pentaricinoleate, Polyglyceryl-6 Pentaricinoleate, Polyglyceryl-10 Pentaricinoleate, Polyglyceryl-4 Pentastearate, Polyglyceryl-6 Pentastearate, Polyglyceryl-10 Pentastearate, Polyglyceryl-3 Polyrisinoleate, Polyglyceryl-6 Polyricinoleate, Polyglyceryl-3 Ricinoleate, Polyglyceryl-2 Sesquiisostearate, Polyglyceryl-2 Sesquioleate, Polyglyceryl-2 Sesquistearate, Polyglyceryl-3 Stearate, Polyglyceryl-2 Stearate, Polyglyceryl-4 Stearate, Polyglyceryl-8 Stearate, Polyglyceryl-10 Stearate, Polyglyceryl-2 Tetraisostearate, Polyglyceryl-6 Tetraoleate, Polyglyceryl-10 Tetraoleate, Polyglyceryl-2 Tetrastearate, Polyglyceryl-2 Triisostearate, Polyglyceryl-3 Triisostearate, Polyglyceryl-10 Trioleate, Polyglyceryl-4 Tristearate, Polyglyceryl Tri stearate, and Polyglyceryl-10 Tristearate.

In some embodiments, at least two polyglyceryl surfactants may include at least one polyglyceryl 3 surfactant and at least one polyglyceryl 6 surfactant.

In some embodiments, at least two polyglyceryl surfactants may include at least one polyglyceryl X stearate type surfactant, such as polyglyceryl X stearate, polyglyceryl X isostearate, polyglyceryl X hydroxystearate, polyglyceryl X distearate, polyglyceryl X diisostearate, polyglyceryl X tristearate, polyglyceryl X triisostearate; polyglyceryl X tetrastearate; polyglyceryl X tetraisostearate, and at least one polyglyceryl Y rinocinoleate, such as polyglyceryl Y polyrinoleate. X and Y each independently may be an integer ranging, for example, from 2 to 12.

In some embodiment, the at least two polyglyceryl surfactants may include at least two surfactants selected from polyglyceryl-6 polyricinoleate, polyglyceryl-3 diisosterate and polyglyceryl-6-polyhydrixystearate.

In some embodiments, the at least two polyglyceryl surfactants may include polyglyceryl-6 polyricinoleate. In some embodiments, the at least two polyglyceryl surfactants may include at least two surfactants polyglyceryl-6 polyricinoleate and polyglyceryl-3 diisosterate. In some embodiments, the at least two polyglyceryl surfactants may include each of polyglyceryl-6 polyricinoleate, polyglyceryl-3 diisosterate and polyglyceryl-6-polyhydroxystearate.

In some embodiments, the at least two polyglyceryl surfactants may comprise Emulium Illustro®, which is polyglyceryl 6-polyhydroxystearate and polyglyceryl-6 polyricinoleate. Emulium Illustro® is commercially available by GATTEFOSSE, France. Surfactants, such as Emulium Illustro®, are disclosed in US-2021/0228458 and WO-2019/239060, each of which is incorporated herein by reference.

In some embodiments, polyglyceryl-6 polyricinoleate may provide the water-in-oil emulsified composition with good stability with no separation between phases and good spreadability.

An amount of the at least two polyglyceryl surfactants in the composition may vary. In some embodiments, the at least two polyglyceryl surfactants may constitute from 0.5 mass % to 15 mass % or from 1 mass % to 15 mass % or from 1 mass % to 10 mass % or 2 mass % to 6 mass % or any value or subrange within these ranges.

In some embodiments, an amount of polyglyceryl rinocinoleate, such as polyglyceryl-6 polyricinoleate may be from 0.3 mass % to 10 mass % or from 0.4 mass % to 6 mass % or from 0.5 mass % to 4 mass % or from 1 mass % to 3 mass % of the composition.

### Solvent

The solvent comprising at least one polar oil and at least one non-polar oil may be a component of the oil phase of the water-in-oil type emulsified composition. An amount of such solvent in the composition may vary. In some embodiments, an amount of such solvent may be from 15 mass % to 50 mass % or from 15 mass % to 40 mass % or from 18 mass % to 35 mass % or from 20 mass % to 30 mass % or any value or subrange within these ranges.

### Polar Oil

The term “polar oil” means any lipophilic (hydrophobic) compound having, at 25° C., a solubility parameter 5_{d} characteristic of dispersive interactions of greater than 16 and a solubility parameter δₚ characteristic of polar interactions of strictly greater than 0. The solubility parameters 5_{d} and δₚ are defined according to the Hansen classification. For example, these polar oils may be chosen from esters, triglycerides and ethers.

The definition and calculation of the solubility parameters in the Hansen three-dimensional solubility space are described in the article by CM. Hansen: “The three dimensional solubility parameters”, J. Paint Technol. 39, 105 (1967).

According to this Hansen space:
5D characterizes the London dispersion forces derived from the formation of dipoles induced during molecular impacts;
δₚ characterizes the Debye interaction forces between permanent dipoles and also the Keesom interaction forces between induced dipoles and permanent dipoles;
5h characterizes the specific interaction forces (such as hydrogen bonding, acid/base, donor/acceptor, etc.); and
5a is determined by the equation: 5ₐ=(δₚ²+5h²)^{1/2}.

The parameters δₚ, 5h, 5p and 5ₐ are expressed in (J/cm³)^{1/2}

The polar oil may be a volatile or non-volatile hydrocarbon-based, silicone and/or fluoro oil.

These oils may be of plant, mineral or synthetic origin.

The term “polar hydrocarbon-based oil” means an oil formed essentially from, or even constituted by, carbon and hydrogen atoms, and optionally oxygen and nitrogen atoms, and not containing any silicon or fluorine atoms. It may contain alcohol, ester, ether, carboxylic acid, amine and/or amide groups.

The term “silicone oil” means an oil containing at least one silicon atom, and especially containing Si—O groups.

The term “fluoro oil” means an oil containing at least one fluorine atom.

The polar oil may have a surface tension of greater than 10 mN/m at 25° C. and at atmospheric pressure.

The surface activity is measured by static tensiometry using the Du Nouy ring.

The principle of the measurement are discussed in WO-2012/110302, which is incorporated by references in its entirety.

In some embodiments, the polar oil may be a non-volatile oil. In particular, the non-volatile polar oil may be chosen from the list of oils below, and mixtures thereof.
hydrocarbon-based polar oils such as phytostearyl esters, such as phytostearyl oleate, phytostearyl isostearate and lauroyl/octyldodecyl/phytostearyl glutamate (Ajinomoto, Eldew PS203), triglycerides consisting of fatty acid esters of glycerol, in particular the fatty acids of which may have chain lengths ranging from C₄ to C36, and especially from C18 to C36, these oils possibly being linear or branched, and saturated or unsaturated; these oils may especially be heptanoic or octanoic triglycerides, wheatgerm oil, sunflower oil, grapeseed oil, sesame seed oil (820.6 g/mol), corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil or musk rose oil; shea butter; or alternatively caprylic/capric acid triglycerides, for instance those sold by the company Stearineries Dubois or those sold under the names Miglyol 810®, 812® and 818® by the company Dynamit Nobel;
synthetic ethers containing from 10 to 40 carbon atoms, such as dicaprylyl ether; -hydrocarbon-based esters of formula RCOOR′ in which RCOO represents a carboxylic acid residue comprising from 2 to 40 carbon atoms, and R′ represents a hydrocarbon-based chain containing from 1 to 40 carbon atoms, such as cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, diisopropyl adipate, heptanoates, and especially isostearyl heptanoate, alcohol or polyalcohol octanoates, decanoates or ricinoleates, for instance propylene glycol dioctanoate, cetyl
octanoate, trioleoyl octanoate, 2-ethylhexyl 4-diheptanoate and palmitate, alkyl benzoate, polyethylene glycol diheptanoate, propylene glycol 2-diethyl hexanoate, and mixtures thereof, C12 to C15 alcohol benzoates, hexyl laurate, neopentanoic acid esters, for instance isodecyl neopentanoate, isotridecyl neopentanoate, isostearyl neopentanoate and 2-octyldodecyl neopentanoate, isononanoic acid esters, for instance isononyl isononanoate, isotridecyl isononanoate and octyl isononanoate, oleyl erucate, isopropyl lauroyl sarcosinate, diisopropyl sebacate, isocetyl stearate, isodecyl neopentanoate, isostearyl behenate, and myristyl myristate;
polyesters obtained by condensation of an unsaturated fatty acid dimer and/or trimer and of diol, such as those described in patent application FR 0 853 634, in particular such as dilinoleic acid and 1,4-butanediol. Mention may especially be made in this respect of the polymer sold by Biosynthis under the name Viscoplast 14436H (INCI name: dilinoleic acid/butanediol copolymer), or else copolymers of polyols and of dimer diacids, and esters thereof, such as Hailuscent ISDA;
polyol esters and pentaerythritol esters, for instance dipentaerythrityl tetrahydroxystearate/tetraisostearate;
fatty alcohols containing from 12 to 26 carbon atoms, for instance octyldodecanol, 2-butyloctanol, 2-hexyldecanol, 2-undecylpentadecanol and oleyl alcohol;
C12-C22 higher fatty acids, such as oleic acid, linoleic acid and linolenic acid, and mixtures thereof;
fluorinated oils which are optionally partially hydrocarbon-based and/or silicone-based;
fatty acids containing from 12 to 26 carbon atoms, for instance oleic acid;
dialkyl carbonates, the two alkyl chains possibly being identical or different, such as dicaprylyl carbonate sold under the name Cetiol CC by Cognis; and
non-volatile oils of high molecular mass, for example between 400 and 10000 g/mol, in particular between 650 and 10000 g/mol, for instance:
   i) vinylpyrrolidone copolymers such as the vinylpyrrolidone/1-hexadecene copolymer, Antaron V-216 sold or manufactured by the company ISP (MW=7300 g/mol),
   ii) esters such as:
   a) linear fatty acid esters with a total carbon number ranging from 35 to 70, for instance pentaerythrityl tetrapelargonate (MW=697.05 g/mol), b) hydroxylated esters such as polyglycerol-2 triisostearate (MW=965.58 g/mol),
   b) aromatic esters such as tridecyl trimellitate (MW=757.19 g/mol), C12-C15 alcohol benzoate, 2-phenylethyl benzoate and butyloctyl salicylate, d) esters of C24-C28 branched fatty acids or fatty alcohols such as those described in patent application EP-A-0 955 039, and especially triisoarachidyl citrate (MW=1033.76 g/mol), pentaerythrityl tetraisononanoate (MW=697.05 g/mol), glyceryl triisostearate (MW=891.51 g/mol), glyceryl tris(2-decyl)tetradecanoate (MW=1143.98 g/mol), pentaerythrityl tetraisostearate (MW=1202.02 g/mol), polyglyceryl-2 tetraisostearate (MW=1232.04 g/mol) or else pentaerythrityl tetrakis(2-decyl)tetradecanoate (MW=1538.66 g/mol),
   e) esters and polyesters of dimer diol and of monocarboxylic or dicarboxylic acid, such as esters of dimer diol and of fatty acid and esters of dimer diol and of dimer dicarboxylic acid, such as Lusplan DD-DA5 and Lusplan DD-DA7© sold by the company Nippon Fine Chemical and described in patent application US 2004-175 338, the content of which is incorporated into the present application by reference,
and mixtures thereof.

In some embodiments, the polar oil is chosen from C12-C15 alcohol benzoate, diisopropyl sebacate, isopropyl lauroyl sarcosinate, dicaprylyl carbonate, 2-phenylethyl benzoate, butyloctyl salicylate, 2-octyldodecyl neopentanoate, dicaprylyl ether, isocetyl stearate, isodecyl neopentanoate, isononyl isononate, isopropyl myristate, isopropyl palmitate, isostearyl behenate, myristyl myristate, octyl palmitate and tridecyl trimellitate.

In some embodiments, the polar oil is a C12-C15 alkyl benzoate.

In some embodiments, the polar oil may be ethylhexyl olivate.

A content of the at least one polar oil in the composition. In some embodiments, an amount of the polar oil, such as ethylhexyl olivate, may be from 4 mass % to 30 mass % or from 5 mass % to 15 mass % or from 25 mass % to 30 mass % or from 8 mass % to 12 mass % or any value or subrange within these ranges.

### Non-Polar Oil

A non-polar oil may one or more of non-polar oils disclosed in U.S. Pat. No. 10,154,954, which is incorporated herein by reference it its entirety.

Nonpolar oils are hydrocarbons. They lack an electronegative element like oxygen, which results in their typical hydrocarbon feel.

These oils may be of vegetable, mineral or synthetic origin.

The term “non-polar oil” may mean an oil for which the solubility parameter at 25 degrees centigrade, δₐ, as defined above, is equal to 0 (J/cm³)_{1/2}.

The term “hydrocarbon oil” may mean an oil formed essentially from, indeed even composed of, carbon and hydrogen atoms, and optionally oxygen and nitrogen atoms, and not containing any silicon or fluorine atoms. It may contain alcohol, ester, ether, carboxylic acid, amine and/or amide groups.

In some embodiments, the non-polar oil may include one or more non-volatile non-polar hydrocarbon oils.

The non-volatile non-polar hydrocarbon oil may be chosen from linear or branched hydrocarbons of mineral or synthetic origin, such as: liquid paraffin or derivatives thereof, squalane, isoeicosane, naphthalene oil, Isosdodecane, isohexadecane; alkane; polybutylenes such as Indopol H-100 (molar mass or MW=965 g/mol), Indopol H-300 (MW=1340 g/mol) and Indopol H-1500 (MW=2160 g/mol) sold or manufactured by the company Amoco, hydrogenated polyisobutylenes such as Parleam® sold by the company Nippon Oil Fats Corporation, Panalane H-300 E sold or manufactured by the company Amoco (MW=1340 g/mol), Viseal 20000 sold or manufactured by the company Synteal (MW=6000 g/mol) or Rewopal PIB 1000 sold or manufactured by the company Witco (MW=1000 g/mol), decene/butene copolymers, polybutene/polyisobutene copolymers, especially Indopol L-14, polydecenes and hydrogenated polydecenes such as: Puresyn 10 (MW=723 g/mol) and Puresyn 150 (MW=9200 g/mol) sold or manufactured by the company Mobil Chemicals, and mixtures thereof.

In some embodiments, the at least one non-polar oil is chosen from hydrogenated polyisobutenes and/or polybutenes.

In some embodiments, the at least one non-polar oil may include squalane (a triterpene consisting of 2,6,10,15,19,23-Hexamethyltetracosane).

In some embodiments, the at least one non-polar oil may include C₉-C₁₄ alkanes, such as isoparaffins, isododecane and isohexadecane. In some embodiments, the at least one non-polar oil may include Vegelight C9-12, which is C₉₋₁₂ Alkanes (and) Coco-caprylate/caprate.

A content of the at least one non-polar oil in the composition may vary. In some embodiments, an amount of the non-polar oil, such as squalane, in the composition may be from 0.5 mass % to 50 mass or from 5 mass % to 25 mass or from 10 mass % to 20 mass % or from 12 mass % to 18 mass % or any value or subrange within these ranges.

In some embodiments, a mass ratio between the non-polar oil, such as squalane, and the polar oil, such as ethylhexyl olivate, may be from 1:100 to 20:1 or 1:60 to 12.5 or from 1:10 to 10:1 or from 1:5 to 1:5 or from 1:3 to 3:1 or from 1:2 to 2:1 or any value or subrange within these ranges.

In some embodiments, a mass ratio between the at least two polyglycerol-type surfactants and the non-polar oil, such as SQUALANE may be from 1:1 to 1:20 or from 1:1 to 1:10 or from 1:1 to 1:5 or from 1:2 to 1:8 or from 1:3 to 1:7 or any value or subrange within these ranges.

Use of a polar oil, such as ethylhexyl olivate, and a non-polar oil, such as squalane may improve adjusting oil polarity between polar and non-polar oils as well as HLB value of a surfactant in the oil phase.

### Thickener

In some embodiments, the composition may comprise at least one thickener. Such thickener may be, for example, at least one hydrophobic mineral gelling agent which may be dissolved in the oily phase in the water-in-oil emulsified composition. For example, the at least one hydrophobic mineral gelling agent may be dissolved in the solvent comprising at least one polar oil and at least one non-polar oil.

The at least one hydrophobic mineral gelling agent may be chosen from but is not limited to organic modified clays and modified or unmodified hectorites and hydrophobic silicas, including fumed silicas. In some embodiments, the at least one hydrophobic mineral gelling agent may be selected from, for example, dimethyl distearyl ammonium hectorite, dimethyl distearyl ammonium bentonite, and dimethyl distearyl ammonium modified montmorillonite and others, as described and exemplified in U.S. Pat. Pub. No. 2007/0071703, which is hereby incorporated by reference.

In some embodiments, the at least one hydrophobic mineral gelling agent may be selected from those in which a quaternary ammonium salt compound is added to a natural or synthetic smectite clay mineral, such as bentonite, by way of an ion exchange reaction. The choice of organic modified clay minerals is not particularly limited as long as it is cosmetically acceptable and may include, for example, dimethyl ammonium hectorite, benzyl dimethyl stearyl ammonium hectorite, and magnesium aluminum silicate treated with distearyl dimethyl ammonium chloride.

Examples of hydrophobic mineral gelling agents include, for example, from bentonites and organically modified hectorites pre-dispersed in organic solvents. A non-limiting example of commercially available bentonite is BENTONE GEL (Registered Trademark) series, including BENTONE GEL (Registered Trademark) ISD V (INCI. Isododecane, Disteardimonium Hectorite, Propylene Carbonate) available from Elementis Specialties. Another exemplary material is GARAMITE 7308XR (INCI: Quatemium-90 Sepiolite and Quaternium-90 Montmorillonite) available from Eckart.

Another applicable hydrophobic mineral gelling agent may comprise silica, in particular fumed silica and those treated with silanol groups. Such hydrophobic silicas are commercially available, for example, under the names of AEROSIL (Registered Trademark) by Degussa and CAB-O-SIL (Registered Trademark) available from Cabot.

Suitable hydrophobic mineral gelling agents may be utilized in a solid powder form or a gel, where the powder(s) are dispersed in a carrier, such as for example mineral oil, isohexadecane, isododecane, hydrogenated polyisobutane, C12-15 alkyl benzoate, and/or isonolnyl isononanoate, polyglyceryl surfactant, such as polyglyceryl-3 diisostarate, a non-polar oil, such as squalane, or a combination thereof.

An amount of the at least one hydrophobic gelling agent, such as bentonite (disterdimonium hectorite), which may be a powder or a gel, may vary. In some embodiments, an amount of the at least one hydrophobic gelling agent, such as bentonite, which may be a power or a gel, may be from 0.1 mass % to 5 mass %.

Use of a hydrophobic gelling agent, such as bentone (disterdimonium hectorite), together with a polar oil, such as ethylhexyl olivate, a polyglyceryl surfactant such as polyglyceryl 3 diisostearate and a non-polar oil, such as squalane, may provide a stable composition system without using a PEG and/or silicon surfactant.

In some embodiments, the at least one hydrophobic gelling agent may comprise a bentone gel, which may be disteardimonium hectorite dispersed in polygyceltyl surfactant, such as polyglyceryl-3 diisostearate, and squalene. An amount of the bentone gel may be from 5 mass % to 15 mass % or from 6 mass % to 10 mass 5 or from 6 mass % to 8 mass % or any value or subrange within these ranges.

### Pigments

In some embodiments, the composition may also include one or more pigments. In some embodiments, one or more pigments may be in a form of a powder. In some embodiments, a powder used as a pigment may be a hydrophobically surface treated powder, which may be dispersed in the oily phase of the composition.

In some embodiments, a powder used as a pigment may be a pigment grade powder. The term “pigment grade” may mean an average particle size in the powder of about 0.2 to 0.4 μm. In some embodiments, one or more pigments may be selected from pigment-grade titanium dioxide, pigment-grade zinc oxide, fine particle zinc oxide, talc, mica, sericite, kaolin, titanated mica, black iron oxide, yellow iron oxide, red iron oxide, ultramarine, Prussian blue, chromium oxide, chromium hydroxide, silica and cerium oxide. In some embodiments, one or more pigments may include one or more mineral pigments, such as one or more iron oxide pigments, such as black iron oxide, yellow iron oxide and red iron oxide. In some embodiments, one or more mineral pigments, such iron oxide pigments, may be hydrophobically surface treated with, for example, lauroyl lysine. Lauroyl lysine treated iron oxide pigments are commercially available, for example, as Unipure Yellow LC 182 LL, Unipure Red LC 381 LL and Unipure Black LC 989 LL.

A content of the one or more pigments in the composition may vary. For example, in some embodiments, an amount of one or more pigments in the composition may be from 0 mass % to 25 mass % or from 0.1 mass % to 25 mass % or from 0.1 mass % to 20 mass % or 0.2 mass % to 15 mass % or from 0.3 mass to 10 mass % or any value or subrange within these ranges.

### Powders

In some embodiments, the composition may include one or more powders. For example, in some embodiments, the one or more powders may be dispersed in the oily phase of the water-in-oil emulsified composition. Yet in some embodiments, the one or more powders may be dispersed in the water phase of the water-in-oil emulsified composition.

In some embodiments, one or more powders may include one or more of titanium dioxide particles, silica particle, iron oxide particles and zinc oxide particles. A hydrophobization surface treatment applied to one or more powders, such as titanium dioxide particles, silica particles, iron oxide particles and/or zinc oxide particles, may be (1) a treatment with a metallic soap consisting of a higher fatty acid and a multivalent metal, such as a divalent, e.g. magnesium, or a trivalent metal, e.g. aluminum or (2) a composite treatment with a higher fatty acid and a hydroxide of a multivalent metal, such as a divalent metal, e.g. magnesium or a trivalent metal, e.g. aluminum. The higher fatty acid may be a C8 to C24, such as C12 to C22, linear or branched carboxylic acid, e.g. stearic acid or isostearic acid. For example, in some embodiments, one or more powders, such as titanium dioxide particles, silica particles iron oxide particles and/or zinc oxide particles, may be surface treated with magnesium stearate and/or magnesium isostearate or with a composite treatment of aluminum hydroxide and stearic and/or isostearic acid.

In some embodiments, one or more powders may provide at least some of sun protection properties, such as ultraviolet protection properties, to the composition. For examples, the one or more powders may comprise one or more ultraviolet scattering powders. Examples of ultraviolet scattering powders include titanium dioxide powders, zinc oxide powders, silica powders, and composite powders, such as titanium dioxide coated mica, titanium dioxide coated bismuth oxychloride, titanium dioxide coated talc and titanium dioxide coated glass flake. Ultraviolet scattering powders may have an average particle diameter of about 25 to 100 nm. In some embodiments, one or more ultraviolet scattering powders may include titatanium dioxide powder, zinc oxide powder or a combination thereof.

Ultraviolet scattering powders may hydrophobic treated on a surface of a base material, such as zinc oxide or titanium dioxide. Examples of methods of hydrophobic treatment of surface include fluorine treatment using perfluoroalkyl phosphoric acid ester, perfluoroalcohol or the like; amino acid treatment using N-acylglutamic acid or the like; lecithin treatment; metallic soap treatment; fatty acid treatment; and alkylphosphoric acid ester treatment.

A content of ultraviolet scattering powder(s) in the composition may vary. In some embodiments, an amount of the ultraviolet scattering powder(s) may be from 2.5 mass % to 30 mass % or from 3 mass % to 25% or from 4 mass % to 20% mass %.

### Organic Ultraviolet Absorbers

In some embodiments, the composition may comprise one or more organic ultraviolet absorbers. In such case, the one or more organic ultraviolet absorbers may provide at least some of sun protection properties, such as ultraviolet protecting properties, to the composition.

The sunlight which reaches the surface of the earth has a fraction of UV-B radiation (280 to 320 nm) and of UV-A radiation (320 to 400 nm) which directly border the visible light region. The effect on the human skin is evident particularly in the case of UV-B radiation through sunburn.

The maximum of the erythema activity of sunlight is given as the relatively narrow range around 308 nm.

To protect against UV-B radiation, numerous compounds are known, which are, inter alia, derivatives of 3-benzylidenecamphor, of 4-aminobenzoic acid, of cinnamic acid, of salicylic acid, of benzophenone, and of 2-phenylbenzimidazole.

It is also important to have available filter substances for the range between about 320 nm and about 400 nm, the so-called UV-A region, since its rays can cause reactions in photosensitive skin. It has been found that UV-A radiation leads to damage of the elastic and collagenous fibers of connective tissue, which causes the skin to age prematurely, and that it should be regarded as the cause of numerous phototoxic and photoallergic reactions. The harmful effect of UV-B radiation can also be intensified by UV-A radiation.

UV photoprotective filters which may be used are oil-soluble organic UV-A filtering compounds and/or UV-B filtering compounds and/or water-soluble UV-A filtering compounds and/or UV-B filtering compounds.

Oil and water soluble UV filtering compounds are disclosed, for example, in U.S. Pat. No. 8,691,196, which is incorporated herein by reference in its entirety.

In some embodiments, one or more organic ultraviolet absorbers oil-soluble organic ultraviolet absorbers, which may be dissolved in the oil phase of the composition. Non-limiting examples of such absorbers include: PABA, PEG-25 PABA, Benzylidenecamphorsulfonic acid, Camphorbenzalkonium methosulfate, Terephthalylidenedicamphorsulfonic acid, Phenylbenzimidazolesulfonic acid, Disodium phenyldibenzimidazoletetrasulfonate, Benzophenone-4, Benzophenone-5, Benzophenone-9, or mixtures thereof.

Examples of oil-soluble ultraviolet absorbers include, but not limited to benzoic acid derivatives, salicylic acid derivatives, cinnamic acid derivatives, dibenzoylmethane derivatives, β-β-diphenyl acrylate derivatives, benzophenone derivatives, benzylidene camphor derivatives, phenylbenzimidazole derivatives, triazine derivatives, phenylbenzotriazole derivatives, anthranil derivatives, imidazoline derivatives, benzal malonate derivatives, and 4,4-diarylbutadiene derivatives.

Examples of benzoic acid derivatives include, but not limited to ethyl p-aminobenzoate (PABA), ethyl dihydroxypropyl PABA, ethylhexyl dimethyl PABA (e.g., “Escalol™. 507; ISP), glyceryl PABA, PEG-25 PABA (e.g., “Uvinul™ P25”; BASF), and diethylamino hydroxybenzoyl hexyl benzoate (e.g., “Uvinul™ A Plus”).

Examples of salicylic acid derivatives include, but not limited to homosalate (“Eusolex™ HMS”; Rona/EM Industries, Inc.), ethylhexyl salicylate (e.g., “Neo Heliopan™ OS”; Haarmann & Reimer), dipropylene glycol salicylate (e.g., “Dipsal™”; Scher), and TEA-salicylate (e.g., “Neo Heliopan™ TS”; Haarmann & Reimer).

Examples of cinnamic acid derivatives include, but not limited to octyl methoxycinnamate or ethylhexyl methoxycinnamate (e.g., “Parsol™ MCX”; Hoffmann-La Roche, Ltd.), isopropyl methoxycinnamate, isoamyl methoxycinnamate (e.g., “Neo Heliopan™ E1000”; Haarmann & Reimer), cinoxate, DEA methoxycinnamate, diisopropyl methylcinnamate, glyceryl ethylhexanoate dimethoxycinnamate, and di-(2-ethylhexyl)-4′-methoxybenzal malonate.

Examples of dibenzoylmethane derivatives include, but not limited to 4-tert-butyl-4′-methoxydibenzoylmethane (e.g., “Parsol™ 1789”).

Examples of β-β-diphenyl acrylate derivatives include octocrylene (e.g., Uvinul™ N539”; BASF).

Examples of benzophenone derivatives include benzophenone-1 (e.g., “Uvinul™ 400”; BASF), benzophenone-2 (e.g., “Uvinul™ D50”; BASF), benzophenone-3 or oxybenzone (e.g., “Uvinul™ M40”; BASF), benzophenone-4 (e.g., “Uvinul™ MS40”; BASF), benzophenone-5, benzophenone-6 (e.g., “Helisorb™ 11”; Norquay Technology Inc.), benzophenone-8 (e.g., “Spectra-Sorb™ UV-24”; American Cyanamid Co.), benzophenone-9 (e.g., “Uvinul™ DS-49”; BASF), and benzophenone-12.

Examples of the benzylidene camphor derivatives include 3-benzylidene camphor (e.g., “Mexoryl™ SD”; Chimex), 4-methylbenzylidene camphor, benzylidene camphor sulfonic acid (e.g., “Mexoryl™ SL”; Chimex), camphor benzalkonium methosulfate (e.g., “Mexoryl™ SO”; Chimex), terephthalylidene dicamphor sulfonic acid (e.g., “Mexoryl™ SX”; Chimex), and polyacrylamide methylbenzylidene camphor (e.g., “Mexoryl™ SW”; Chimex).

Examples of phenylbenzimidazole derivatives include phenylbenzimidazole sulfonic acid (e.g., “Eusolex™ 232”; Merck KGaA), and disodium phenyl dibenzimidazole tetrasulfonate (e.g., “Neo Heliopan™ AP”; Haarmann & Reimer).

Examples of triazine derivatives include anisotriazine (e.g., “Tinosorb™ S”; Ciba Specialty Chemicals Inc.), ethylhexyl triazone (e.g., “Uvinul™ T-150”; BASF), diethylhexyl butamido triazone (e.g., “Uvasorb™ HEB”; 3V SIGMA S.p.A.), and 2,4,6-tris(diisobutyl-4′-aminobenzalmalonate)-s-triazine.

Examples of phenylbenzotriazole derivatives include drometrizole trisiloxane (e.g., “Silatrizole™ Rhodia Chimie), and methylene bis-benzotriazolyl tetramethylbutylphenol (e.g., “Tinosorb™ M”; Ciba Specialty Chemicals Inc.).

Examples of anthanil derivatives include menthyl anthranilate (e.g., “Neo Heliopan™ MA”; Haarmann & Reimer).

Examples of imidazoline derivatives include ethyihexyl dimethoxybenzylidene dioxoimidazoline propionate.

Examples of the benzal malonate derivatives include polyorganosiloxane having a benzal malonate functional group (e.g., Polysilicone-15; “Parsol™ SLX”; DSM Nutrition Japan K.K.).

Examples of the 4,4-diarylbutadiene derivatives include 1,1-dicarboxy(2,2′-dimethylpropyl)-4,4-diphenylbutadiene.

A content of the one or more organic ultraviolet absorbers, such as one or more oil soluble ultraviolet absorbers, may vary. In some embodiments, the composition may contain no such absorbers at all. In such case, sun protection properties of the composition may be due to ultraviolet scattering powder(s). Yet in some embodiments, the composition may contain the one or more organic ultraviolet absorbers, such as one or more oil soluble ultraviolet absorbers, in an amount from 1 mass % to 25 mass % or from 3 mass % to 20 mass % or 5 mass % to 18 mass %

### Exemplary Composition

In some embodiments, the composition may comprise from 1 mass % to 5 mass % of polyglyceryl-6 polyricinoleate; from 1 mass % to 5 mass % of at least one polyglyceryl-3 diisosterate and polyglyceryl-6-polyhydrixystearate; from 6 mass % to 10 mass % bentone gel; from 8 mass % to 12 mass % of ethylhexyl olivate; and from 12 mass % to 18 mass % of squalene.

### Additional Ingredients

In some embodiments, the composition may include one or more additional ingredients, such as a preservative, such as phenoxyethanol; a preservative boosting surfactant, such as propanediol; humectant, an emollient and/or a moisturizer, such as glycerin, a polysaccaride, such as amylopectin.

Yet in some embodiments, the composition may contain the one or more the additional ingredients in an amount from 1 mass % to 25 mass % or from 3 mass % to 20 mass % or 5 mass % to 18 mass %.

### Method of Making

The composition may be prepared by preparing a homogenized oil phase; and adding the water phase to an oil phase and performing further homogenizing of the product.

In some embodiments, the process may involve adding one or more additional phase into the oil phase before it is added to the water phase. The addition product of the oil phase and the additional phase may be also homogenized before being added to the water phase.

The additional phase may be for example, a pigment phase formed from hydrophobic pigments.

All the steps of preparing the composition may be performed at a temperature from 15 C to 32 C or from 18 C to 30 C or from 20 C to 28 C or at room temperature, such as a temperature about 25 C.

Embodiments described herein are further illustrated by, though in no way limited to, the following working examples.

### Example

TABLE 1
Summary of selected compositions

| | | | | Example A | Example B | Example C |
|---|---|---|---|---|---|---|
| BONTANESSENTIAL | ETHYLHEXYL OLIVATE, | 27.5 | 17.5 | 27.5 (1.35%) | 10.5 (0.25%) | 10 (0.5%) |
| OLIVE S-EHO | SQUALANE*(5%) | | | | | |
| PLUROL | POLYGLYCERYL 3- | 3 | 3 | 0 | 0 | 3 |
| DIISOSTERARIS | DIISOSTEARATE | | | | | |
| INHOUSE BETONE | S-HECTORITE, | 5 | 15 | 7 | 7 | 7 |
| GEL S-EHO | POLYGLYCEREL-3 | | | | | |
| | DISTEARDIMONIUM | | | | | |
| | DIISOSTEARTE, | | | | | |
| | SQUALENE* | | | | | |
| EMULIUM | POLYGLYCERYL 6- | | | 3 | 3 | |
| ILLUSTRO | POLYHYDROXYSTEARATE | | | | | |
| | AND POLYGLYCEROL-6 | | | | | |
| | POLYRICINOLEATE | | | | | |
| BARSERSE-15 | POLYGLYCEROL-6 | | | | | 1.5 |
| | POLYRICINOLEATE | | | | | |
| NEOSSANCE | SQUALANE* | | | | 15 | 14 |
| SQUALANE | | | | | | |
| EVALUATION | | NO | NOT | GOOD | MOST | MOST |
| (visual) | | GOOD | PERFECT | (slight | STABLE | STABLE |
| | | (separated) | | separated) | (no separation) | (no separation) |

*contribute to a total amount of squalane in the compositions.

The data in Table 1 indicates that to stabilize a composition, an appropriate content of bentone gel may be selected together with an appropriate emulsifier.

TABLE 2

| | | | | AIC INNOVATION & | |
|---|---|---|---|---|---|
| | | AIC INNOVATION & | | SUSTAINABILITY | |
| | | SUSTAINABILITY | | Comparative | |
| | | Example 1 | | Example 1 | |
| Raw Material | INCI Name | INVENTIVE | | CONTROL | |
| DEIONIZED WATER | WATER | 35.23 | | 35.18 | |
| TAYCA MP-25 MG | TITANIUM DIOXIDE & | 10.99 | | 10.99 | |
| | MAGNESIUM STEARATE | | | | |
| BOTANESSENTIAL | ETHYLHEXYL OLIVATE & | 10.5 | (9.975:0.525) | 27.5 | (26.125:1.375) |
| OLIVE S-EHO | SQUALANE* (95:5) | | | | |
| NEOSSANCE ™ | SQUALANE* | 10.00 | | | |
| SQUALANE | | | | | |
| BM SQUALANE- | (ETHYLHEXYL OLIVATE & | 7 | (4.9:1.4:0.7) | 5 | (3.5:1:0.5) |
| EHO BENTONE GEL | SQUALANE*) &DISTEARDIMONIUM | | | | |
| | HECTORITE & POLYGLYCERYL-3 | | | | |
| | DIISOSTEARATE (70:20:10) | | | | |

| VEGELIGHT | C9-12 ALKANE & COCO- | 5.00 | | |
|---|---|---|---|---|
| C912-LC | CAPRYLATE/CAPRATE | | | |
| SEPIFINE ™ BB | AMYLOPECTIN | 3.50 | | 3.50 |
| EMULIUM ® | POLYGLYCERYL-6 | 3 | (1.387:1.387:0.225) | |
| ILLUSTRO | POLYHYDROXYSTEARATE & | | | |
| | POLYGLYCERYL-6 | | | |
| | POLYRICINOLEATE & | | | |
| | POLYGLYCERIN-6 & TOCOPHEROL | | | |
| | & ASCORBYL PALMITATE | | | |
| | (46.235:46.235:7.5:0.20:0.01) | | | |

| | | | |
|---|---|---|---|
| TITANIUM DIOXIDE | TITANIUM DIOXIDE & ALUMINUM | 3.00 | 3.00 |
| MT-100 TVS | HYDROXIDE & STEARIC ACID | | |
| ZEMEA | PROPANEDIOL | 3.00 | 3.00 |
| UNIPURE YELLOW | IRON OXIDES & LAUROYL LYSINE | 2.16 | 2.16 |
| LC 182 LL | | | |
| AQUA CACTEEN | GLYCERIN & WATER & OPUNTIA | 2.00 | 2.00 |
| | FICUS-INDICA STEM EXTRACT | | |
| | & PHENOXYETHANOL | | |
| GLYCYLGLYCINE | DIPEPTIDE-15 | 1.60 | 1.60 |
| ATOLIGOMER | SEA SALT | 1.00 | 1.00 |
| PHENOXETOL | PHENOXYETHANOL | 0.97 | 0.97 |
| NF 272556 | | | |
| UNIPURE RED | IRON OXIDES & | 0.65 | 0.65 |
| LC 381 LL | LAUROYL LYSINE | | |
| (PULVERIZED) | | | |
| UNIPURE BLACK | IRON OXIDES & | 0.20 | 0.20 |
| LC 989 LL | LAUROYL LYSINE | | |
| (PULVERIZED) | | | |
| EDTA 3•NA2•H20 | TRISODIUM EDTA | 0.20 | 0.20 |
| PLUROL | POLYGLYCERYL-3 | | 3.00 |
| DIISOSTEARIQUE | DIISOSTEARATE | | |
| CG #5793 | | | |
| NATROSOL | HYDROXYETHYLCELLULOSE | | 0.05 |
| 250 HHR PC | | | |

*Contributes to Total amount of Squalane.

Example 1 from Table 2 corresponds to Inventive Example B from Table 1.

Control Example 1 from Table 2 corresponds to Comparative Example A from Table 1.

Compositions of Examples in Table 1 and 2 were prepared using by the following cold process. Phase A (Oil phase) was prepared by homogenizing ingredients for phase A for 5-10 min or until uniform at room temperature. Ingredients of phase B (pigments phase) were added to the main batch of phase A and homogenized for 20 min. The homogenized product was checked for color dispersion. The temperature was kept at room temperature level.

Phase C (water phase) was prepared in a separate vessel by add water and the rest of ingredients for phase C using a propeller mixer at room temperature (RT).

Phase C was added to the homogenization product of phases A and B and homogenized for 10 min at room temperature (RT).

Compositions of Comparative Examples were prepared by the following hot process.

Phase A (Oil phase) was prepared by homogenizing ingredients for phase A for 5-10 min or until uniform at room temperature. Ingredients for phase B (pigments phase) were added to the main batch of phase A at room temperature and homogenizes for 20 min. The homogenized product was checked for color dispersion. The homogenization product of phases A and B was heated to 50-55° C.

Phase C (water phase) was prepared in a separate vessel by add water and the rest of ingredients for phase C using a propeller mixer at 70-75° C. The mixture was heat to 50-55° C. The heated phase C was added the heated homogenized product of phases A and B and homogenized for 10 min at 50-55° C. Then the temperature was lowered to 45° C.

Phase D was added to the heated homogenized product of phases A, B and C at 45° C. The product was homogenized for additional 5 min.

### Sensory Test

The objective was to evaluate and compare the composition of Example 1 and the control composition of Comparative Example 1 in a sensory study. Both compositions had a neutral ivory shade. Evaluation protocol: 5 panelists evaluated the two compositions in a 2-day study, all products were blinded. The following use instructions (self-application) were provided for each panelist: apply with fingers/applicator onto face. Use product 1 on Day 1, use product 2 on Day 2. Evaluate face throughout the day and answer the questionnaire as directed. Evaluation: Sensory Questionnaire: answer the questions by analyze the characteristic of product using the provide scale from 0-10. Higher number is better performance.

TABLE 3
Results of evaluation of the composition of Example 1 and the control composition of Comparative Example 1.

| | | | | | | | | | | Skin | | |
|---|---|---|---|---|---|---|---|---|---|---|---|---|
| | | Blend- | Play | | | Powdery | Natural | Smooth- | Added | Feels | Light | |
| | Sticky | ability | Time | Coverage | Evenness | Look | Look | ness | Moisture | Tight | weight | Radiant |
| Example 1 | 8.2 | 9.8 | 3.8 | 6.2 | 9.2 | 3.8 | 8.4 | 9.2 | 7.2 | 8.8 | 7.2 | 7.6 |
| Comparative | 7.8 | 9.4 | 3 | 5.2 | 8.8 | 3.2 | 7.4 | 7.8 | 8 | 8.6 | 7.6 | 7.8 |
| Example 1 | | | | | | | | | | | | |

Conclusion: the composition of Example 1 and the control composition of Comparative Example 1 were similar in all categories but the composition of Example 1 was slightly smoother than the composition of Comparative Example 1.

TABLE 4
stability test results for the composition of Example 1 and the control composition of Comparative Example 1.

| | WK1 | | | | WK2 | | | | WK3 | | | | WK4 | | | |
|---|---|---|---|---|---|---|---|---|---|---|---|---|---|---|---|---|
| | 25C | 45C | 50C | M1 | 25C | 45C | 50C | M1 | 25C | 45C | 50C | M1 | 25C | 45C | 50C | M1 |
| Comparative | − | − | − | − | − | − | − | − | − | − | − | − | − | − | − | − |
| Example 1 | | | | | | | | | | | | | | | | |
| Example 1 | + | + | + | + | + | + | + | + | + | + | + | + | + | + | + | + |

For the stability test 0.2 oz glass jar samples of the compositions of Example 1 and Comparative Example 1 were placed at 25° C., 45° C., 50° C. and −5-45° C. (M1) Stability, including odor, appearance and viscosity, was checked weekly. Freeze thaw test: 2 cycles (−10 C to 25 C). Sample placed in −10 C chamber for 48 hrs. Moved to 25 C chamber for 48 hrs. Check stability. Repeated for another cycle. (Was freeze thaw test repeated every week?)

In Table 4, “−”: Failed stability test—oil separation and great deviation in appearance. “+”: Passed stability rest—no oil separation, the product is stable.

Conclusion: the composition of Comparative Example 1 showed oil separation and white striation at room temperature after 3 days. The composition of Comparative Example 1 failed the freeze thaw test after 1 cycle. The composition of Example 1 showed no oil separation, no white striation after 4 weeks at any of the used temperatures. The composition of the composition of Example 1 passed the freeze thaw test.

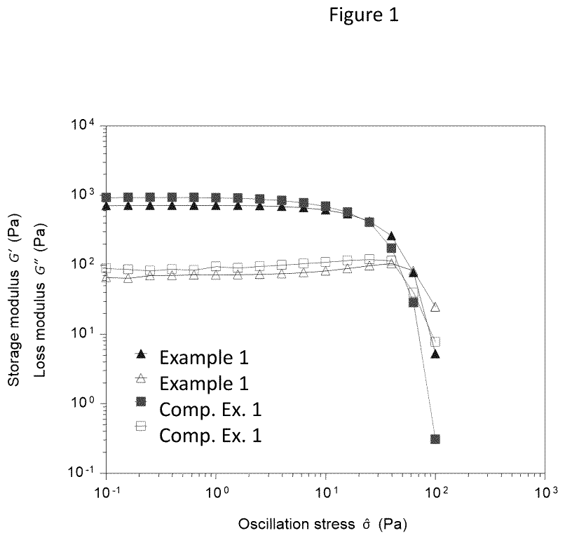

FIG. 1 presents plots of storage modulus (G′) and loss modulus (G″) versus oscillation stress for the composition of Example 1 (triangles) and the control composition of Comparative Example 1 (squares). The data in FIG. 1 allows evaluating yield stress, which is indicative of structural strength, for each of the compositions. The yield stress for Example 1 was 61.02 Pa, which is 16.3% greater than the yield stress for Comparative Example 1.

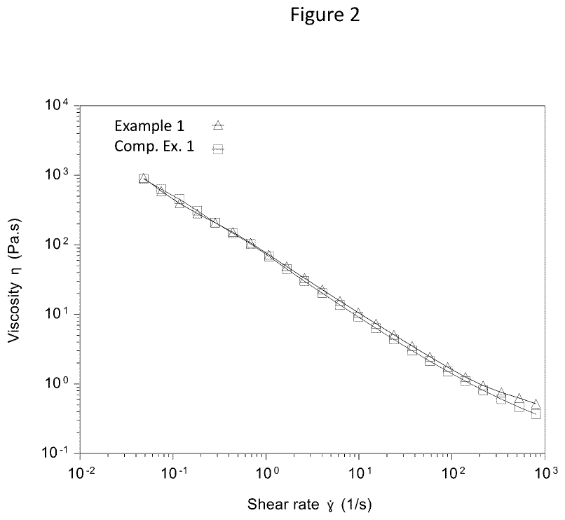

FIG. 2 presents plots of viscosity versus shear rate for the composition of Example 1 (triangles) and the control composition of Comparative Example 1 (squares). Table 5 shows shear viscosity values at 4 l/s and 90 l/s.

TABLE 5

Viscosity (Pa · s)

| | 4 (1/s) | 90 (1/s) |
|---|---|---|
| Example 1 | 22.51 | 1.70 |
| Comparative Example 1 | 19.91 | 1.49 |

TABLE 6
Exemplary Foundation Composition

| Phase | RM Name | EU INCI | Supplier | RM % |
|---|---|---|---|---|
| A | EMULIUM ® | POLYGLYCERYL-6 POLYHYDROXYSTEARATE, | GATTEFOSSE USA | 3.00 |
| | ILLUSTRO | POLYGLYCERYL-6 POLYRICINOLEATE, | | |
| | | POLYGLYCERIN-6, TOCOPHEROL, | | |
| | | ASCORBYL PALMITATE | | |
| A | BOTANESSENTIAL | ETHYLHEXYL OLIVATE, SQUALANE | BOTANIGENICS, INC | 10.50 |
| | OLIVE S-EHO | | | |
| | NEOSSANCE ™ | SQUALANE | CENTERCHEM | 15.00 |
| | SQUALANE | | | |
| A | BM SQUALANE-EHO | ETHYLHEXYL OLIVATE, DISTEARDIMONIUM | SHISEIDO | 7.00 |
| | BENTONE GEL | HECTORITE, POLYGLYCERYL-3 | COMPANY LTD. | |
| | | DIISOSTEARATE, SQUALANE | | |
| B | TITANIUM DIOXIDE | TITANIUM DIOXIDE (nano), ALUMINUM | SUMITOMO SHOJI | 3.00 |
| | MT-100 TVS | HYDROXIDE, STEARIC ACID | CHEMICALS | |
| B | TAYCA MP-25 MG | CI 77891, MAGNESIUM | PRESPERSE | 10.99 |
| | | STEARATE | CORPORATION | |
| B | UNIPURE YELLOW | CI 77492, LAUROYL LYSINE | SENSIENT COSMETIC | 2.16 |
| | LC 182 LL | | TECHNOLOGIES | |
| B | UNIPURE RED | CI 77491, LAUROYL LYSINE | DAVLYN | 0.65 |
| | LC 381 LL | | INDUSTRIES | |
| | (PULVERIZED) | | | |
| B | UNIPURE BLACK | CI 77499, LAUROYL LYSINE | DAVLYN | 0.20 |
| | LC 989 LL | | INDUSTRIES | |
| | (PULVERIZED) | | | |
| C | DEIONIZED WATER | AQUA | SUPPLIER NOT | 35.18 |
| | | | SPECIFIED | |
| C | NATROSOL | HYDROXYETHYLCELLULOSE | SHISEIDO | 0.05 |
| | 250 HHR PC | | COMPANY LTD. | |
| C | EDTA 3•NA2•H20 | TRISODIUM EDTA | SHISEIDO | 0.20 |
| | | | COMPANY LTD. | |
| C | GLYCYLGLYCINE | DIPEPTIDE-15 | SHISEIDO | 1.60 |
| | | | COMPANY LTD. | |
| C | ATOLIGOMER | MARIS SAL | BARNET PRODUCTS | 1.00 |
| | | | CORPORATION | |
| C | SEPIFINE ™ BB | AMYLOPECTIN | SEPPIC S.A. | 3.50 |
| C | ZEMEA | PROPANEDIOL | DUPONT TATE | 3.00 |
| | | | AND LYLE | |
| C | AQUA CACTEEN | GLYCERIN, AQUA, OPUNTIA FICUS- | MIBELLE AG | 2.00 |
| | | INDICA STEM EXTRACT, PHENOXYETHANOL | BIOCHEMISTRY | |
| C | PHENOXETOL | PHENOXYETHANOL | CLARIANT | 0.97 |
| | NF 272556 | | CORPORATION | |
| | | | | 100 |

TABLE 7
Another exemplary foundation composition

| Phase | RM Name | EU INCI | Supplier | RM % |
|---|---|---|---|---|
| A | BARSPERSE-15 | POLYGLYCERYL-6 POLYRICINOLEATE | BARNET PRODUCTS | 1.5 |
| | | | CORPORATION | |
| A | PLUROL | POLYGLYCERYL-3 DIISOSTEARATE | GATTEFOSSE USA | 3 |
| | DIISOSTEARIQUE | | | |
| | CG #5793 | | | |
| A | BOTANESSENTIAL | ETHYLHEXYL OLIVATE, SQUALANE | BOTANIGENICS, INC | 10 |
| | OLIVE S-EHO | | | |
| A | NEOSSANCE ™ | SQUALANE | CENTERCHEM | 14 |
| | SQUALANE | | | |
| A | BM SQUALANE-EHO | ETHYLHEXYL OLIVATE, | SHISEIDO | 7 |
| | BENTONE GEL | DISTEARDIMONIUM HECTORITE, | COMPANY LTD. | |
| | | POLYGLYCERYL-3 | | |
| | | DIISOSTEARATE, SQUALANE | | |
| B | TITANIUM DIOXIDE | TITANIUM DIOXIDE (nano), | SUMITOMO SHOJI | 3 |
| | MT-100 TVS | ALUMINUM HYDROXIDE, | CHEMICALS | |
| | | STEARIC ACID | | |
| B | TAYCA MP-25 MG | CI 77891, MAGNESIUM | PRESPERSE | 10.99 |
| | | STEARATE | CORPORATION | |
| B | UNIPURE YELLOW | CI 77492, LAUROYL LYSINE | SENSIENT COSMETIC | 2.16 |
| | LC 182 LL | | TECHNOLOGIES | |
| B | UNIPURE RED | CI 77491, LAUROYL LYSINE | DAVLYN | 0.65 |
| | LC 381 LL | | INDUSTRIES | |
| | (PULVERIZED) | | | |
| B | UNIPURE BLACK | CI 77499, LAUROYL LYSINE | DAVLYN | 0.2 |
| | LC 989 LL | | INDUSTRIES | |
| | (PULVERIZED) | | | |
| C | DEIONIZED WATER | AQUA | SUPPLIER NOT | 35.18 |
| | | | SPECIFIED | |
| C | NATROSOL | HYDROXYETHYLCELLULOSE | SHISEIDO | 0.05 |
| | 250 HHR PC | | COMPANY LTD. | |
| C | EDTA 3•NA2•H20 | TRISODIUM EDTA | SHISEIDO | 0.2 |
| | | | COMPANY LTD. | |
| C | GLYCYLGLYCINE | DIPEPTIDE-15 | SHISEIDO | 1.6 |
| | | | COMPANY LTD. | |
| C | ATOLIGOMER | MARIS SAL | BARNET PRODUCTS | 1 |
| | | | CORPORATION | |
| C | SEPIFINE ™ BB | AMYLOPECTIN | SEPPIC S.A. | 3.5 |
| D | ZEMEA | PROPANEDIOL | DUPONT TATE | 3 |
| | | | AND LYLE | |
| D | AQUA CACTEEN | GLYCERIN, AQUA, OPUNTIA | MIBELLE AG | 2 |
| | | FICUS-INDICA STEM | BIOCHEMISTRY | |
| | | EXTRACT, PHENOXYETHANOL | | |
| D | PHENOXETOL | PHENOXYETHANOL | CLARIANT | 0.97 |
| | NF 272556 | | CORPORATION | |
| | | | | 100 |

TABLE 8
Exemplary concealer composition

| Phase | RM Name | EU INCI | Supplier | RM % |
|---|---|---|---|---|
| A | PLUROL | POLYGLYCERYL-3 | GATTEFOSSE USA | 2 |
| | DIISOSTEARIQUE | DIISOSTEARATE | | |
| | CG #5793 | | | |
| A | BARSPERSE-15 | POLYGLYCERYL-6 | BARNET PRODUCTS | 1 |
| | | POLYRICINOLEATE | CORPORATION | |
| A | KOBOGUARD | HYDROGENATED | KOBO PRODUCTS | 15 |
| | 5400 SQ | POLYCYCLOPENTADIENE | | |
| | | AND SQALANE | | |
| A | VEGELIGHT | C9-12 ALKANE, COCO- | GRANT INDUSTRIES | 10 |
| | C912-LC | CAPRYLATE/CAPRATE | | |
| A | AEROSIL ® | SILICA | EVONIK | 1.25 |
| | 200 PHARMA | | GOLDSCHMIDT | |
| | | | CORPORATION | |
| B | TAYCA MP-25 MG | CI 77891, MAGNESIUM | PRESPERSE | 16.04 |
| | | STEARATE | CORPORATION | |
| B | UNIPURE YELLOW | CI 77492, LAUROYL LYSINE | SENSIENT COSMETIC | 4.57 |
| | LC 182 LL | | TECHNOLOGIES | |
| B | UNIPURE RED | CI 77491, LAUROYL LYSINE | DAVLYN | 0.96 |
| | LC 381 LL | | INDUSTRIES | |
| | (PULVERIZED) | | | |
| B | UNIPURE BLACK | CI 77499, LAUROYL LYSINE | DAVLYN | 0.43 |
| | LC 989 LL | | INDUSTRIES | |
| | (PULVERIZED) | | | |
| C | BM SQUALANE-EHO | ETHYLHEXYL OLIVATE, | SHISEIDO | 12 |
| | BENTONE GEL | DISTEARDIMONIUM HECTORITE, | COMPANY LTD. | |
| | | POLYGLYCERYL-3 | | |
| | | DIISOSTEARATE, SQUALANE | | |
| C | AMIHOPE LL | LAUROYL LYSINE | AJINOMOTO NORTH | 1 |
| | | | AMERICA INC | |
| C | MSS-500W | SILICA | KOBO PRODUCTS | 2 |
| D | DEIONIZED WATER | AQUA | SUPPLIER NOT | 25.58 |
| | | | SPECIFIED | |
| D | GLYCERINE NAT | GLYCERIN | BRENNTAG | 3 |
| | 99.7% ACIDCHEM | | NORTHEAST LLC | |
| D | EDTA 3•NA2•H20 | TRISODIUM EDTA | SHISEIDO | 0.2 |
| | | | COMPANY LTD. | |
| D | ATOLIGOMER | MARIS SAL | BARNET PRODUCTS | 1 |
| | | | CORPORATION | |
| D | ZEMEA | PROPANEDIOL | DUPONT TATE | 3 |
| | | | AND LYLE | |
| D | PHENOXETOL | PHENOXYETHANOL | CLARIANT | 0.97 |
| | NF 272556 | | CORPORATION | |
| | | | | 100 |

TABLE 9
Another Exemplary Concealer Composition

| Phase | RM Name | EU INCI | Supplier | RM % |
|---|---|---|---|---|
| | EMULIUM ® | POLYGLYCERYL-6 | GATTEFOSSE USA | 3.00 |
| | ILLUSTRO | POLYHYDROXYSTEARATE, | | |
| | | POLYGLYCERYL-6 POLYRICINOLEATE, | | |
| | | POLYGLYCERIN-6, TOCOPHEROL, | | |
| | | ASCORBYL PALMITATE | | |
| A | KOBOGUARD | HYDROGENATED | KOBO PRODUCTS | 15.00 |
| | 5400 SQ | POLYCYCLOPENTADIENE | | |
| A | VEGELIGHT | C9-12 ALKANE, COCO- | GRANT INDUSTRIES | 10.00 |
| | C912-LC | CAPRYLATE/CAPRATE | | |
| A | AEROSIL ® | SILICA | EVONIK | 1.25 |
| | 200 PHARMA | | GOLDSCHMIDT | |
| | | | CORPORATION | |
| B | TAYCA MP-25 MG | CI 77891, MAGNESIUM | PRESPERSE | 16.04 |
| | | STEARATE | CORPORATION | |
| B | UNIPURE YELLOW | CI 77492, LAUROYL LYSINE | SENSIENT COSMETIC | 4.57 |
| | LC 182 LL | | TECHNOLOGIES | |
| B | UNIPURE RED | CI 77491, LAUROYL LYSINE | DAVLYN | 0.96 |
| | LC 381 LL | | INDUSTRIES | |
| | (PULVERIZED) | | | |
| B | UNIPURE BLACK | CI 77499, LAUROYL LYSINE | DAVLYN | 0.43 |
| | LC 989 LL | | INDUSTRIES | |
| | (PULVERIZED) | | | |
| C | BM SQUALANE-EHO | ETHYLHEXYL OLIVATE, | | 12.00 |
| | BENTONE GEL | DISTEARDIMONIUM HECTORITE, | | |
| | | POLYGLYCERYL-3 | | |
| | | DIISOSTEARATE, SQUALANE | | |
| C | AMIHOPE LL | LAUROYL LYSINE | AJINOMOTO NORTH | 1.00 |
| | | | AMERICA INC | |
| C | MSS-500W | SILICA | KOBO PRODUCTS | 2.00 |
| D | DEIONIZED WATER | AQUA | SUPPLIER | 25.58 |
| | | | NOT SPECIFIED | |
| D | GLYCERINE NAT | GLYCERIN | BRENNTAG | 3.00 |
| | 99.7% ACIDCHEM | | NORTHEAST LLC | |
| D | EDTA 3•NA2•H20 | TRISODIUM EDTA | SHISEIDO | 0.20 |
| | | | COMPANY LTD. | |
| D | ATOLIGOMER | MARIS SAL | BARNET PRODUCTS | 1.00 |
| | | | CORPORATION | |
| D | ZEMEA | PROPANEDIOL | DUPONT TATE | 3.00 |
| | | | AND LYLE | |
| D | PHENOXETOL | PHENOXYETHANOL | CLARIANT | 0.97 |
| | NF 272556 | | CORPORATION | |
| | | | | 100.00 |

Concealer compositions such the ones in Tables 8-9 may be prepared using the following cold process: Phase A (Oil phase) may be prepared by Homogenizing ingredients for phase A for 5-10 min or until uniform at room temperature. Ingredients for phase B (pigments phase) may be added to the main batch and homogenizes for 20 min. The product of phases A and B is checked for color dispersion. The product of phases A and B is kept at room temperature.

Ingredients of phase C are added to the product of phases A and B one by one and homogenized for 5-10 min. The product of phases A, B and C is kept at room temperature.

Phase D (water phase) is prepared in a separate vessel by add water and the rest of ingredients for phase D using a propeller mixer at room temperature. Phase D is added to the product of phases A, B and C and homogenized for 10 min at room temperature.

Although the foregoing refers to particular preferred embodiments, it will be understood that the present disclosure is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A water-in-oil type emulsified cosmetic composition comprising:
(a) at least two polyglycerol-type surfactants;
(b) a solvent comprising at least one polar oil and at least one non-polar oil; and
(c) water, which constitutes no more that 40% by mass of the composition.

2. The cosmetic composition of claim 1, wherein the at two polyglycerol-type surfactants comprise at least two of polyglyceryl-6 polyricinoleate, polyglyceryl-3 diisosterate and polyglyceryl-6-polyhydrixystearate.

3. The cosmetic composition of claim 1, wherein the at two polyglycerol-type surfactants comprise polyglyceryl-6 polyricinoleate

4. The cosmetic composition of claim 1, wherein the at least two polyglyceryl-type surfactants constitute from 1 mass % to 15 mass % of the composition.

5. The cosmetic composition of claim 1, wherein the at least one polar oil comprise ethylhexyl olivate and the at least one non-polar oil comprises squalene.

6. The cosmetic composition of claim 1, wherein the solvent constitutes from 15 mass % to 40 mass % of the composition.

7. The composition of claim 1, wherein the at least one non-polar oil constitutes from 10 mass % to 20 mass % of the composition.

8. The composition of claim 1, wherein a mass ratio between the at least two polyglycerol-type surfactants and the at least one non-polar oil is from 1:1 to 1:10.

9. The composition of claim 1, wherein the composition further comprises a thickener comprising bentonite.

10. The composition of claim 8, wherein the bentonite constitutes from 6 mass % to 10 mass % of the composition.

11. The composition of claim 1, further comprising one or more pigments.

12. The composition of claim 1, further comprising at least one powder.

13. The composition of claim 12, wherein the at least one powder comprises at least one UV scattering powder.

14. The composition of claim 13, wherein the at least one UV scattering powder comprises at least one of zinc oxide powder or titanium oxide powder.

15. The composition of claim 12, wherein the at least one powder has a hydrophobically treated surface.

16. The composition of claim 1, further comprising at least one UV-absorbing organic molecule.

17. The composition of claim 1, comprising from 1 mass % to 5 mass % of polyglyceryl-6 polyricinoleate; from 1 mass % to 5 mass % of at least one polyglyceryl-3 diisosterate and polyglyceryl-6-polyhydrixystearate; from 6 mass % to 10 mass % bentonite; from 8 mass % to 12 mass % of ethylhexyl olivate; and from 12 mass % to 18 mass % of squalene.

18. A cosmetic method comprising applying the cosmetic composition of claim 1 to a keratinous surface.
